(19) United States
(12) Patent Application Publication
(10) Pub. No.: US 2022/0401180 A1
(43) Pub. Date: Dec. 22, 2022

(54) Specimen Marking Clip

(71) Applicant: MarginView, LLC, Denver, CO (US)

(72) Inventor: David Opperman, Littleton, CO (US)

(73) Assignee: MarginView, LLC, Denver, CO (US)

(21) Appl. No.: 17/805,716

(22) Filed: Jun. 7, 2022

Related U.S. Application Data
(60) Provisional application No. 63/213,168, filed on Jun. 21, 2021.

(51) Int. Cl. A61B 90/00

(52) U.S. Cl. CPC A61B 90/39; A61B 2090/3966; A61B 2090/3991; A61B 2090/3983

(57) ABSTRACT

A specimen marking clip is provided that is adapted to selectively attach to tissue inside of a patient and corresponding tissue that has been excised from the patient for analysis. Sutures may be associated with the clips to help ensure correct in vivo and ex vivo sample orientation. In vivo clips may remain in the patient's body if necessary.

This application claims the benefit of U.S. Provisional Patent Application Ser. No. 63/213,168, filed Jun. 21, 2021, the entirety of which is incorporated by reference herein.

The Appendix included is incorporated by reference herein.

This application is related to pending U.S. patent application Ser. No. 16/711,653, filed Dec. 12, 2019, which is a continuation-in-part of abandoned U.S. patent application Ser. No. 16/162,017, filed Oct. 16, 2018, which is a continuation-in-part of U.S. patent application Ser. No. 15/139,012, filed Apr. 26, 2016, now U.S. Pat. No. 10,111,727, issued Oct. 30, 2018, which claims the benefit of U.S. Provisional Patent Application Ser. No. 62/162,035, filed May 15, 2015, the entire disclosures of which are incorporated by reference herein.

### FIELD OF THE INVENTION

Embodiments of the present invention are generally related to medical devices, more particularly, surgical specimen margin marking and orientation devices and methods.

### BACKGROUND OF THE INVENTION

Surgical specimen margin marking to define sample orientation for endoscopic, robotic, laparoscopic, or other surgery types where body tissue is removed is critical for pathological diagnosis, tumor excision, etc. Specimen margin orientation marking is often needed for endoscopic excision of a tumor. In such an example, the tumor is removed and forwarded to a pathologist for evaluation. The pathologist subsequently performs an analysis to identify malignancy in the sample. A surgeon may then direct additional tissue to be excised, the orientation of which is critical for proper and accurate excision.

The current surgical specimen margin marking technique uses ink to mark an excised specimen. One conventional method comprises marking a specimen mass with a line to differentiate it from tissue surrounding an excised specimen mass. The ink mark may be placed on the specimen mass at a “12:00 o'clock” position. This approach is inaccurate and subject to error if the orientation of the specimen is changed (e.g., dropped, manipulated, etc.) before marking. The surgeon, staff, or pathologist examining the specimen mass may also mistakenly manipulate the removed specimen mass before marking, or there may be miscommunication between surgeons and pathologists related to orientation. Further, it may be difficult to find the ink marks and compare them to those on the removed specimen mass. An alternative approach includes placing a reference suture in the specimen at the 12:00 o'clock position. However, this approach is equally subject to error.

Accordingly, an improved surgical specimen margin orientation marking method is desired. The systems and methods described herein allow for mirror image marking of multiple types of surgical specimens covering a multitude of surgeries and disciplines.

### SUMMARY OF INVENTION

It is one aspect of some embodiments of the present invention to provide a marking clip configured to selectively engage tissue. The marking clip of one embodiment includes first and second legs extending from a common interconnection point. Opposite ends of the legs are spaced but can be selectively deformed toward each other when an external force is applied to the outside surfaces of the legs. In operation, tissue is placed between the legs and the clip is deformed, thereby pinching the tissue. In one embodiment, portions of the legs include teeth or a roughened/knurled surface that enhances grip. The legs and/or the common interconnection point may be more malleable to facilitate clip deformation.

A biopsied area of interest, e.g., a possibly cancerous mass, is removed from a patient's body along with a predefined amount of surrounding tissue. “Margin,” as used herein, is the peripheral edge of the removed sample tissue, defined by the incision created to remove the tissue sample. Those of ordinary skill in the art will appreciate that accurate analysis, which is needed to ensure all abnormal tissue has been removed from the patient's body, is important and relies on maintaining tissue sample orientation. That is, understanding the orientation of the tissue sample relative to the excision location helps the surgeon and pathologist identify additional areas of concern on the tissue sample so that the surgeon can remove corresponding tissue from the patient.

Accordingly, it is another aspect of some embodiments to provide a clip that maintains the in vivo and ex vivo orientation of an excised tissue sample. More specifically, each clip of some embodiments of the present invention is of a unique color assigned to a predefined tissue sample attachment location on the margin. To further ensure proper post-excision tissue sample orientation, embodiments of the present invention interconnect like color clips to corresponding margin locations within the patient's body. One of ordinary skill in the art will appreciate that other means can be used to match ex vivo and in vivo clips.

Those of ordinary skill in the art will appreciate that other identification techniques may be used to define clip location on the tissue sample and in vivo. One embodiment, for example, employs colored sutures interconnected to otherwise indistinct clips. Other embodiments contemplate colored clips having sutures of matching colors extending therefrom. Alternatively, sutures positioned on one side of a margin (e.g., tissue sample side) may have one color/indicia and sutures positioned on the opposite side of the margin (e.g., in vivo side) may have a second color/indicia, thereby allowing quick identification of which suture to attach relative to the margin. Sutures may be striped with clip color to indicate their intended attachment location—in vivo or tissue sample side.

The clips may be made of material that suits the tissue to which they are to be attached—muscle, ligament, skin, fatty tissue, etc. The clips can be of any size and shape and made of any suitable material. For example, the clip's material of manufacture may be stiff, compliant, malleable, etc., to suit the surgeon's desires or to accommodate tissue character. In one embodiment, the clips are made of titanium per ASTM F67 Grade 1. The clips may possess a surface texture or other types of tactile or enhanced visual identification means. Further, the clips may be micro-engraved or X-ray marked with identifying information. The sutures also may employ microchip technology, nanotech technology, RFID technology, specialized coatings, frequency emitting devices that allow current or future-developed 3-dimensional, computer, or virtual reality medical imaging modalities to locate and identify sutures in the body. These marking methods may be provided in combination or in various sub-combinations.

The sutures of some embodiments of the present invention are stiff, compliant, malleable, etc., to suit the surgeon's desires. The sutures of one embodiment are made of PTFE coated braided polyester, meeting all requirements established by the United States Pharmacopedia (U.S.P) for nonabsorbable surgical sutures. The sutures may possess a surface texture or other types of tactile or enhanced visual identification means. Further, the sutures may be micro-engraved or X-ray identifiable with identifying information. The sutures also may employ microchip technology, nanotech technology, RFID technology, specialized coatings, frequency emitting devices that allow current or future-developed 3-dimensional, computer, or virtual reality medical imaging modalities to locate and identify sutures in the body. These marking methods may be provided in combination or in various sub-combinations.

In one example, the clips and/or sutures are configured to selectively deliver medication to the patient. That is, the aspects of various embodiments of the present invention may also allow for the utilization of adjuvant therapies that employ selective and/or controlled application of drugs, radiation, etc. In one embodiment, the clips/sutures left in the patient's body employ treatment means, e.g., drug and/or radiation delivery systems. The clips/sutures of some embodiments employ drug and/or radiation delivery systems or are constructed at least partially from such materials that provide treatment over an extended period of time. For example, all or a portion(s) of the clips/sutures may comprise a drug-infused co-polymer (i.e., a polymer drug conjugate) manufactured of polylactide-polyglycolide similar to that found in dissolvable sutures.

The suture or clip may have diagnostic qualities, wherein a characteristic change occurs when conditions around the specimen mass change. For example, tumor growth, tissue DNA/RNA change, etc., would initiate a color change in the clips/sutures. The clips/sutures may also possess the ability to transmit diagnostic information outside the patient's body.

In another example, the clips and/or sutures are configured to indicate special information, e.g., the distance between clips or sutures, the distance between a clip and the specimen mass within the body, etc., which may assist in ascertaining mass growth. One of ordinary skill in the art will appreciate that the clips may employ other types of indicia alone or in combination with corresponding indicia of the sutures without departing from the scope of the embodiments of the present invention. Further, the characteristics of the clips and/or sutures do not have to match identically across the margin boundary; the primary concern is that one must later be able to orient the mass relative to the area of excision, which will be described below correctly.

The surgeon may implement the clip/suture combination as an effective tool for manipulating tissues, which is more efficient than attaching a handle to an otherwise slippery surface. For example, the sutures and/or clips described herein can facilitate traction, counter traction, specimen mass control during robotic and laparoscopic cases, and lift the specimen mass into extraction bags. The clips/sutures contemplated herein may also be used to facilitate wound closure or as anchor points for implants.

The Summary of the Invention is neither intended nor should it be construed as being representative of the full extent and scope of the present invention. That is, these and other aspects and advantages will be apparent from the disclosure of the invention(s) described herein. Further, the above-described embodiments, aspects, objectives, and configurations are neither complete nor exhaustive. As will be appreciated, other embodiments of the invention are possible using, alone or in combination, one or more of the features set forth above or described below. Moreover, references made herein to “the present invention” or aspects thereof should be understood to mean certain embodiments of the present invention and should not necessarily be construed as limiting all embodiments to a particular description. The present invention is set forth in various levels of detail in the Summary of the Invention as well as in the attached drawings and the Detailed Description and no limitation as to the scope of the present invention is intended by either the inclusion or non-inclusion of elements, components, etc. in this Summary of the Invention. Additional aspects of the present invention will become more readily apparent from the Detailed Description, particularly when taken together with the drawings.

The above-described benefits, embodiments, and/or characterizations are not necessarily complete or exhaustive, and in particular, as to the patentable subject matter disclosed herein. Other benefits, embodiments, and/or characterizations of the present invention are possible utilizing, alone or in combination, as set forth above and/or described in the accompanying figures and/or in the description herein below.

The phrases “at least one,” “one or more,” and “and/or,” as used herein, are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

Unless otherwise indicated, all numbers expressing quantities, dimensions, conditions, and so forth used in the specification and drawing figures are to be understood as being approximations which may be modified in all instances as required for a particular application of the novel assembly and method described herein.

The term “a” or “an” entity, as used herein, refers to one or more of that entity. As such, the terms “a” (or “an”), “one or more” and “at least one” can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

It shall be understood that the term “means” as used herein shall be given its broadest possible interpretation in accordance with 35 U.S.C., Section 112(f). Accordingly, a claim incorporating the term “means” shall cover all structures, materials, or acts set forth herein, and all of the equivalents thereof. Further, the structures, materials, or acts and the equivalents thereof shall include all those described in the Summary, Brief Description of the Drawings, Detailed Description and in the appended drawing figures.

### BRIEF DESCRIPTION OF THE DRAWINGS

The accompanying drawings, which are incorporated in and constitute a part of the specification, illustrate embodiments of the invention and together with the general description of the invention given above and the detailed description of the drawings given below, serve to explain the principles of these inventions.

FIG. 1 is a perspective view of the clip of one embodiment of the present invention.

FIG. 2 is a front elevation view of the clip shown in FIG. 1.

FIG. 3 is a detailed view of FIG. 2.

FIG. 4 is a detailed view of FIG. 2

FIG. 5 the top plan view of the clip shown in FIG. 1.

FIG. 6 is a detailed view of FIG. 5.

FIG. 7 is a right elevation view of the clip shown in FIG. 1.

FIG. 8 is a perspective view of the clip system of one embodiment of the present invention.

FIG. 9 is a front elevation view of the clip system illustrating the removal of one clip from a housing.

FIG. 10 is a representation showing the first step in removing a tissue sample from the body.

FIG. 11 is a representation showing the incorporation of clips to an in vivo side and a margin side of the tissue sample.

FIG. 12 is a representation showing removal of the tissue sample from a patient.

FIG. 13 is a representation showing the removed sample and the incorporation of a deep margin clip in vivo.

FIG. 14 illustrates both sides of the removed sample.

FIG. 15 is a representation of mapping sample's area concerned to a corresponding in vivo location.

FIG. 16 is a representation showing a defined second margin that surrounds a new area of concern.

FIG. 17 is a representation showing a removed second sample and the incorporation of a second deep margin clip.

FIG. 18 shows an example of a report concerning the clips of one embodiment of the present invention.

The following component list and associated numbering found in the drawings is provided to assist in the understanding of one embodiment of the present invention:
# Component
2 Clip
6 First leg
10 Second leg
14 Proximal end
18 First leg distal end
22 Second leg distal end
26 Teeth
30 Tapered portion
34 Suture hole
38 Clip system
42 Base
46 Housing
48 Adhesive
50 Suture
54 Applier
58 Margin
62 Periphery
66 Mass
70 Sample
74 In vivo tissue
78 Cavity
82 Underside
86 New area of concern
90 New margin
94 Report
R Red
G Green
B Blue
Y Yellow
GR Gray
P Purple
i In vivo side
s Sample side
n New

It should be understood that the drawings are not necessarily to scale. In certain instances, details that are not necessary for an understanding of the invention or that render other details difficult to perceive may have been omitted. It should be understood, of course, that the invention is not necessarily limited to the particular embodiments illustrated herein.

### DETAILED DESCRIPTION

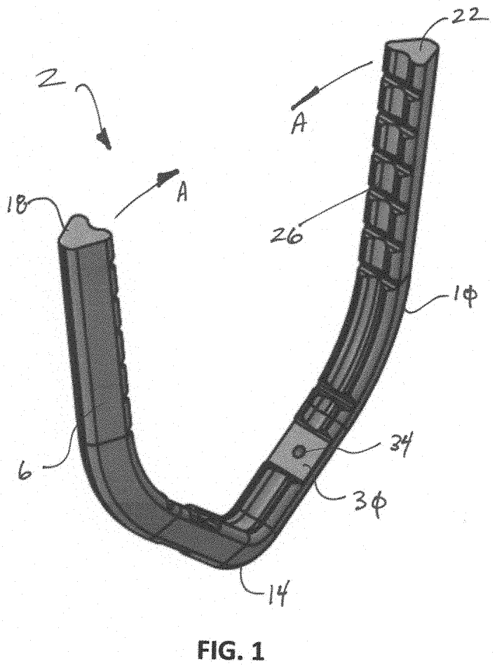

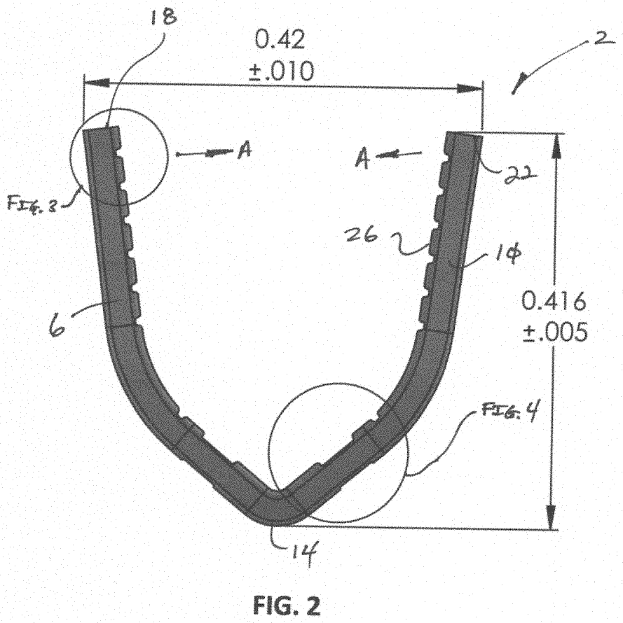

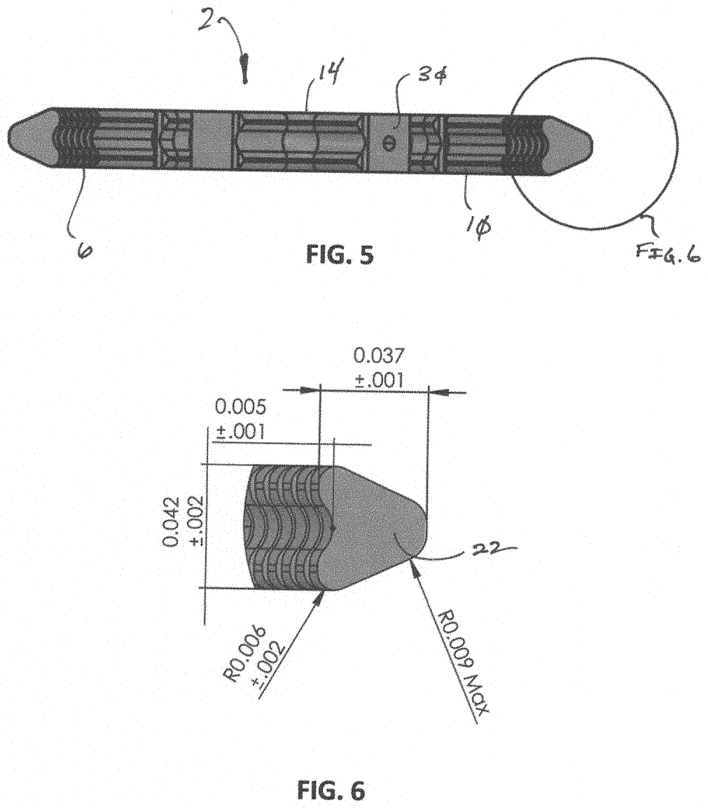

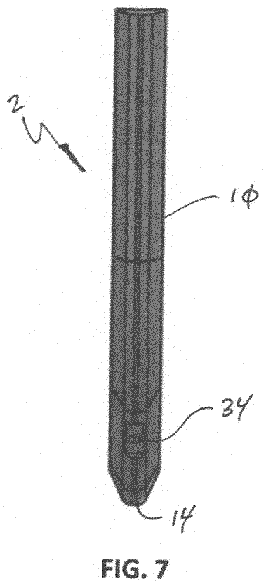

FIGS. 1-7 show clip 2 of one embodiment of the present invention comprised of a first leg 6 and the second leg 10. The first leg 6 and the second leg 10 have operatively interconnected proximal ends 14 and corresponding distal ends 18 and 22. In one embodiment, the proximal ends are interconnected by a living hinge, a hinge, or a deformable member. The deformable members may be of a thickness less than that of the legs to allow the distal ends to be urged towards each other. In some embodiments, the deformable portions are spaced from the leg's proximal ends. Further, those of ordinary skill in the art will appreciate that the legs are inherently deformable. In operation, pressure applied to the outside surface of the first and/or second leg will selectively deform the clip 2 and move the distal end of the first leg 18 and the distal end of the second leg 22 towards each other, generally in the direction of Arrow A. One of ordinary skill in the art will appreciate that the dimensions shown in FIGS. 2-4 and 6 are applicable to one embodiment of the present invention and, thus, are for reference only.

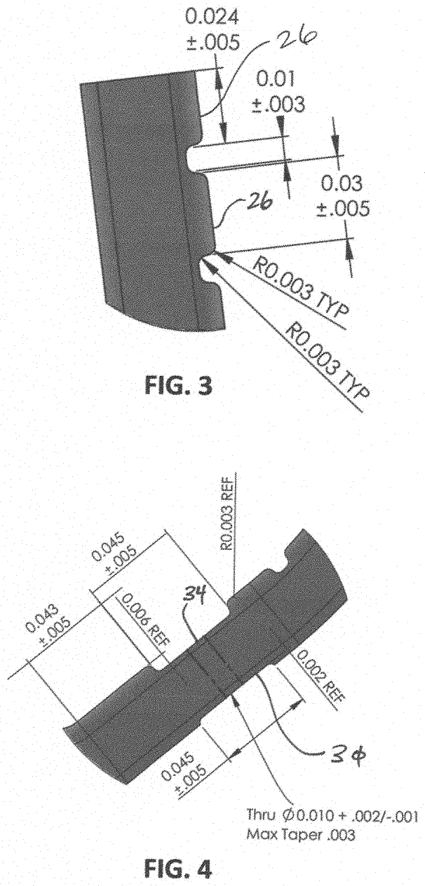

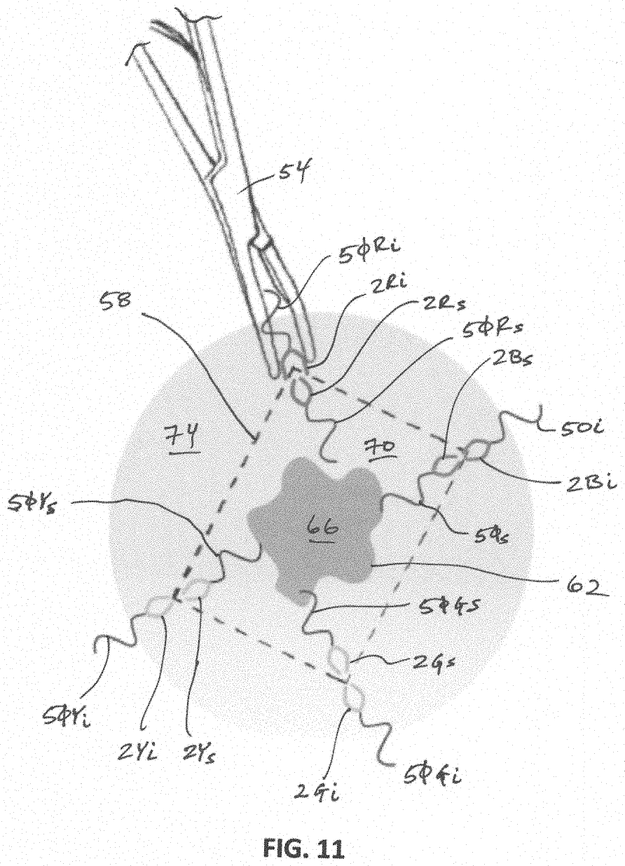

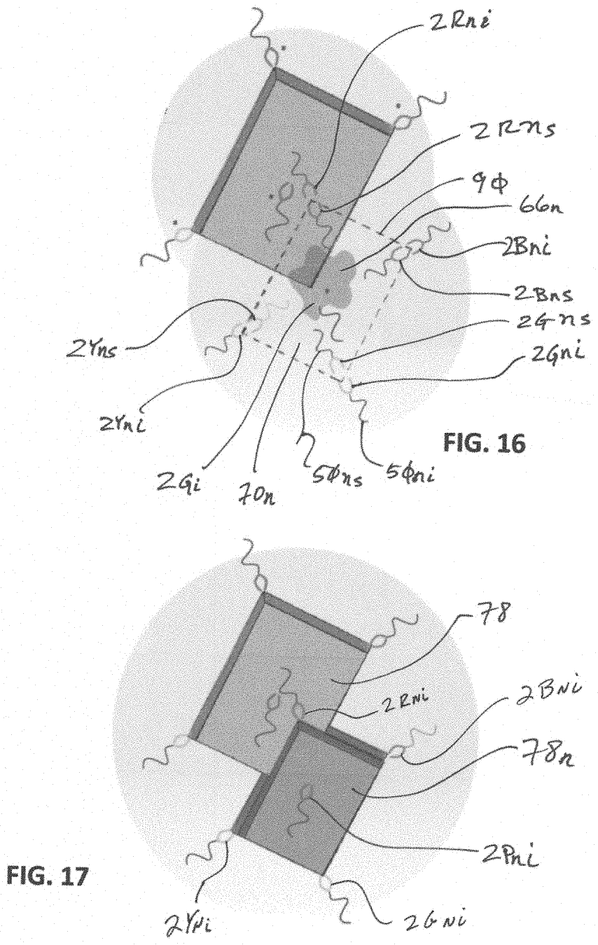

FIGS. 2 and 3 highlight the nature of the inner surfaces of the first and second legs of one embodiment, wherein teeth 26 are provided to facilitate clip grasping onto a tissue sample taken from a patient's body and/or in vivos tissue associated with the excised sample, which will be apparent upon review of FIGS. 11-17. In operation, biasing of the legs towards each other will bring corresponding teeth 26 into close proximity with tissue therebetween.

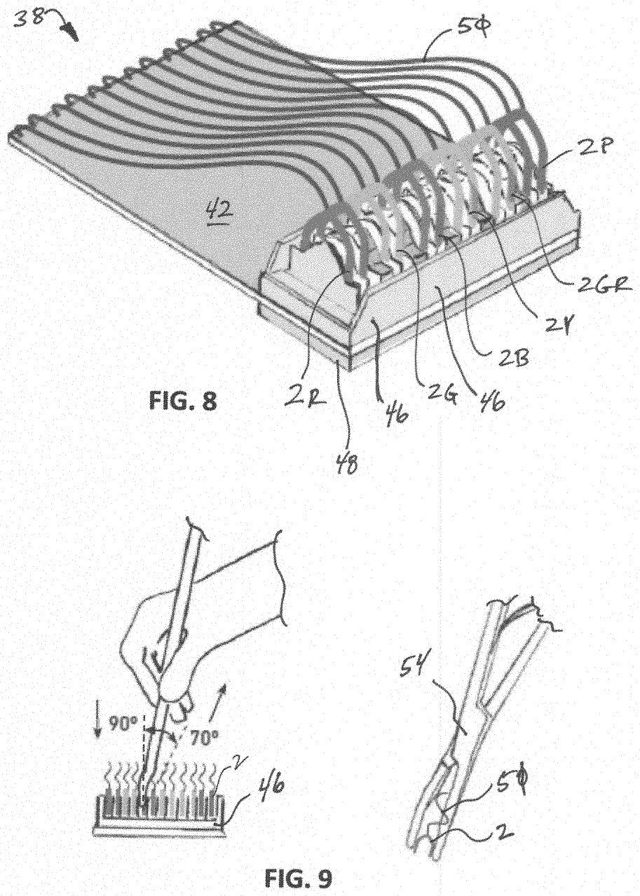

FIG. 4 shows another portion of the clip adjacent to the proximal end 14. Here, a tapered portion 30 is provided that accommodates a suture hole 34. The suture hole 34 receives a suture as shown in FIG. 8 that helps identify the clip. The tapered section 30 may also facilitate movement of the distal ends towards each other.

FIG. 8 shows a clip system 38 of one embodiment of the present invention having a base 42 with interconnected housing 46 that accommodates a plurality of clips 2 and associated sutures 50. For example, adhesive 48 can be used to secure the base 42 to a tray. In this example, colored suture pairs consisting of margin side and in vivo side are shown, wherein the colors denote predefined approximate locations of clip placement, which will be described in further detail below. As mentioned above, suture color may denote margin side or in vivo side or match the clip color. One of ordinary skill in the art will appreciate that although solid colors are alluded to herein, other identification methods, such as stripes or striations, may be employed without departing from the scope of the invention.

FIG. 9 illustrates the removal of a clip 2 from the housing. Here, the housing includes cavities that selectively receive and securely maintain the clips within the housing. This feature ensures clip sterility and decreases the chances of the incorrect clip being removed because the physician must make a concerted effort to remove a desired clip from the housing. An applier 54 is used to grasp and angulate the clip 2 a predetermined amount, in the example shown about 20° from horizontal, to initiate clip removal. Thereafter, the clip 2 remains held by the applier 54 in an orientation suitable for interconnection to tissue.

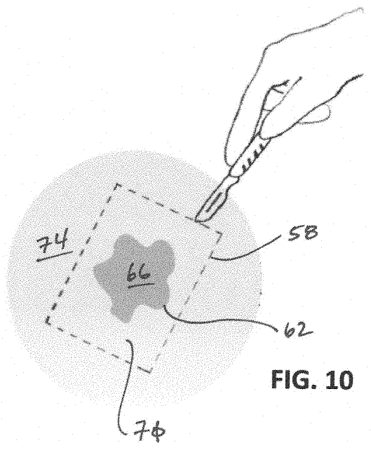

FIGS. 10 and 11 illustrate pre-sample removal from the patient. The first step in the operation is to define a margin 58 a predetermined distance from a periphery 62 of a mass 66, i.e., the area of concern being biopsied. The margin 58, thus, defines a sample 70 that will be removed from the patient. Before removal, however, a plurality of clips are associated with the sample 70 and in vivo tissue 74 associated with the sample. According to one embodiment, color-coded clips 2Rs, 2Bs, 2Gs, and 2Ys indicate locations on the sample (s), and corresponding color-coded clips 2Ri, 2Bi, 2Gi, and 2Yi indicate mirrored, in vivo (i) locations. One of skill in the art will appreciate the sample side clips 2s and the in vivo side clips 2i may have the same color or otherwise be identified as a pair with predefined indicia (e.g., A and 1, B and 2, etc.), integrated RFID technology, x-ray markers, etc.

Color-coated sutures can be provided on the sample side 50Rs, 50Bs, 50Gs, and 50Ys and the in vivo side 50Ri, 50Bi, 50Gi, and 50Yi. Suture color or marking method may correspond with the interconnected clip color or marking method. Alternatively, suture color may depend on whether the clip is on the sample side 50s or the in vivo side 50i. In FIG. 11, the sutures are of the same color. In one embodiment, red clamps/sutures indicate an anterior sample location. Similarly, green, blue, purple, and yellow clamps/sutures represent inferior, superior, posterior, and deep locations, respectively. However, other embodiments may feature various other color-coding schemes.

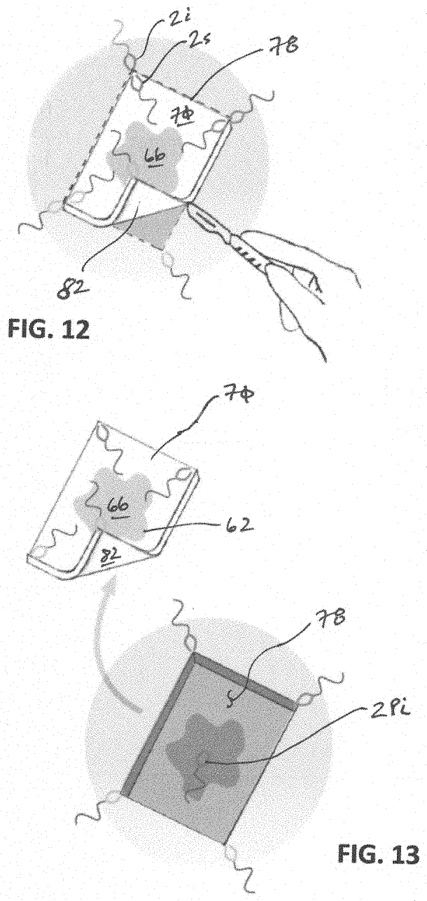

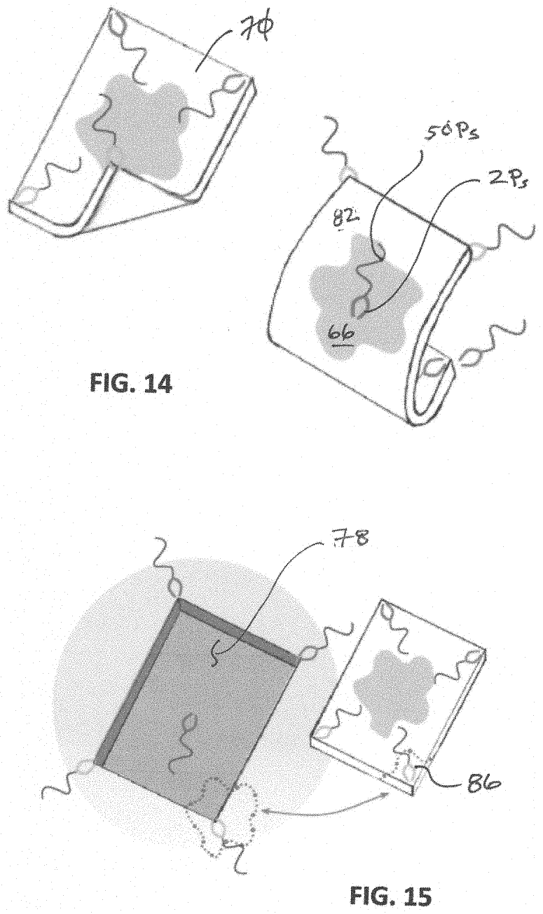

In operation, a surgeon will attach one marking clip 2s to the margin 58 associated with a predefined portion of the specimen mass 66, and a second marking clip 2i to tissue 74 surrounding the specimen mass 66, outside the margin 58. Subsequently, an incision is made along the margin 58, which is shown in FIGS. 10 and 12. The result is a mirror image marking of the two areas—sample and in vivo tissue. The paired clips allow pathologists to quickly match the orientation of the specimen (ex-vivo) relative to a cavity 78 created (in-vivo). FIGS. 13 and 14 show a deep-tissue clip 2Pi placed within the cavity 78, wherein the corresponding sample clip 2Ps is placed on the underside 82 of the sample.

FIGS. 15-17 illustrate one aspect of some embodiments of the present invention that helps a pathologist identify a new area of concern 86. The new area of concern 86 defines a new margin 90, in the example shown, associated with the green clip 2Gs. Accordingly, the surgeon can create an accurate new margin 90 because the corresponding in vivo green clip 2Gi can be quickly located. A series of new clips 2Rni, 2Rns, 2Bni, 2Bns, 2Gni, 2Gns, 2Yni, 2Yns, 2Pni, and 2Pns (not shown) can then be associated with a new tissue sample (not shown) and the new cavity 78n. As one of ordinary skill in the art will appreciate, the process of defining new margins will continue until the pathologist is satisfied that all areas of concern have been excised.

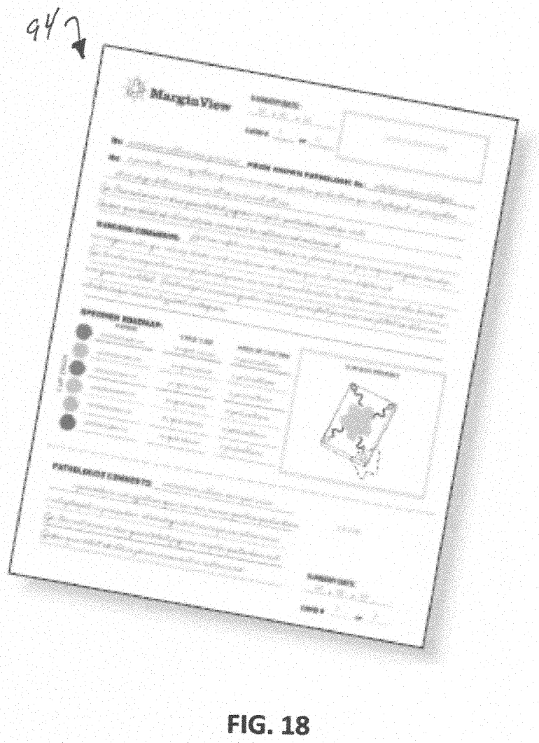

FIG. 18 is a representation of a report 94 associated with the biopsy or mass removal procedure. The report 94 is designed to provide a historical record of the entire surgical procedure. If the area of concern becomes an issue in the future, the nature of the in vivo clips can be assessed and compared with the prior procedure. For example, if the area of concern shifts to a new clip set, the pathology of the mass may be assessed and amended to provide a new strategy for addressing the medical issue. The report 94 will provide a detailed description of color and clip location for immediate or future use.

Exemplary characteristics of embodiments of the present invention have been described. However, to avoid unnecessarily obscuring embodiments of the present invention, the preceding description may omit several known apparatus, methods, systems, structures, and/or devices one of ordinary skill in the art would understand are commonly included with the embodiments of the present invention. Such omissions are not to be construed as a limitation of the scope of the claimed invention. Specific details are set forth to provide an understanding of some embodiments of the present invention. It should, however, be appreciated that embodiments of the present invention may be practiced in a variety of ways beyond the specific detail set forth herein.

Modifications and alterations of the various embodiments of the present invention described herein will occur to those skilled in the art. It is to be expressly understood that such modifications and alterations are within the scope and spirit of the present invention, as set forth in the following claims. Further, it is to be understood that the invention(s) described herein is not limited in its application to the details of construction and the arrangement of components set forth in the preceding description or illustrated in the drawings. That is, the embodiments of the invention described herein are capable of being practiced or of being carried out in various ways. The scope of the various embodiments described herein is indicated by the following claims rather than by the foregoing description. And all changes which come within the meaning and range of equivalency of the claims are to be embraced within their scope. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The foregoing disclosure is not intended to limit the invention to the form or forms disclosed herein. In the foregoing Detailed Description, for example, various features of the invention are grouped together in one or more embodiments for the purpose of streamlining the disclosure. This method of disclosure is not to be interpreted as reflecting an intention that the claimed inventions require more features than expressly recited. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention. Further, the embodiments of the present invention described herein include components, methods, processes, systems, and/or apparatus substantially as depicted and described herein, including various sub-combinations and subsets thereof. Accordingly, one of skill in the art will appreciate that would be possible to provide for some features of the embodiments of the present invention without providing others. Stated differently, any one or more of the aspects, features, elements, means, or embodiments as disclosed herein may be combined with any one or more other aspects, features, elements, means, or embodiments as disclosed herein.

What is claimed is:

1. A clip adapted to selectively interconnect to tissue, comprising:
a first leg having a proximal end with a plurality of inwardly-extending teeth and a distal end;
a second leg having a proximal end with a plurality of inwardly-extending teeth and a distal end;
wherein the first leg and the second leg are curved, and wherein the proximal end of the first leg and the proximal end of the second end define an apex, wherein the first and second legs extend away from the apex in the same direction; and
a tapered portion on the first leg or the second leg, wherein the tapered portion is configured to facilitate selective leg deformation.

2. The clip of claim 1, further comprising a suture interconnected to at least one of the first leg or the second leg.

3. The clip of claim 1, wherein at least one of the suture or clip includes identifying indicia.

4. The clip of claim 3, wherein the identifying indicia comprises at least one of a surface texture, a micro-engraving, x-ray markings, microchip technology, nanotech technology, RFID chips, and a coating.

5. The clip of claim 1, wherein the distal ends of the first leg and the second end are spaced such that a distance between the distal ends is less than a length of the first leg or the second leg.

6. A clip adapted to selectively interconnect to tissue, comprising:
a first leg having a proximal end and a distal end;
a second leg having a proximal end and a distal end;
wherein the first leg and the second leg are curved, and wherein the proximal end of the first leg and the proximal end of the second end define an apex, wherein the first and second legs extend away from the apex in the same direction; and
a tapered portion on the first leg or the second leg, wherein the tapered portion is configured to facilitate selective leg deformation.

7. The clip of claim 6, further comprising a suture interconnected to at least one of the first leg or the second leg.

8. The clip of claim 7, wherein the clip and suture are of the same color.

9. The clip of claim 7, wherein at least one of the suture or clip includes identifying indicia.

10. The clip of claim 9, wherein the identifying indicia comprises at least one of a surface texture, a micro-engraving, x-ray markings, microchip technology, nanotech technology, RFID chips, and a coating.

11. The clip of claim 6, wherein the distal ends of the first leg and the second end are spaced such that a distance between the distal ends is less than a length of the first leg or the second leg.

12. A method of marking a tissue specimen removed from a patient, comprising:
identifying an area of interest in the patient's body;
defining the outer boundary of the area of interest;
defining a margin having a perimeter spaced from the outer boundary of the area of interest;
attaching a tissue sample clip between the area of interest and the margin;
attaching an in vivo clip outside the margin and opposite to the tissue sample clip;
creating an incision around the area of interest that generally corresponds with the margin, the incision defining a boundary of a tissue sample;
removing the tissue sample and interconnected tissue sample clip; and
maintaining the in vivo clip within the patient's body.

13. The method of claim 12, further comprising interconnecting a deep tissue in vivo clip in a cavity that remains in the patient's body when the tissue sample is removed, and interconnecting a corresponding deep tissue sample clip to the tissue sample.

14. The method of claim 13, wherein the tissue sample clip and the in vivo clip are of a first color, and the deep tissue in vivo clip and deep sample clip are of a second color.

15. The method of claim 12, further comprising:
analyzing the tissue sample and identifying a second area of interest;
using the clips to map the second area of interest to a corresponding location in the patient's body;
defining a second margin around the corresponding location in the patient's body;
attaching a second tissue sample clip between the corresponding location in the patient's body and the second margin;
attaching a second in vivo clip outside the second margin and opposite the second tissue sample clip; and
creating a second incision around the corresponding location in the patient's body, generally corresponding with the second margin, the second incision defining a boundary of a second tissue sample.

16. The method of claim 12, wherein the tissue sample clip and in vivo clip each comprise:
a first leg having a proximal end and a distal end;
a second leg having a proximal end and a distal end;
wherein the first leg and the second leg are curved, and wherein the proximal end of the first leg and the proximal end of the second end define an apex, wherein the first and second legs extend away from the apex in the same direction; and
a tapered portion on the first leg or the second leg, wherein the tapered portion is configured to facilitate selective leg deformation.

17. The method of claim 16, further comprising a suture interconnected to at least one of the first leg or the second leg.

18. The method of claim 17, wherein the tissue sample clip, the suture associated with the tissue sample clip, the in vivo clip, and the suture associated with the in vivo clip are of the same color.

19. The method of claim 17, wherein the tissue sample clip and the in vivo clip are of the same color, the suture associated with the tissue sample clip has a first identifying indicia, and the suture associated with the in vivo clip has a second identifying indicia.

20. The method of claim 19, wherein the first indicia and second indicia are different.
